(19) United States
(12) Patent Application Publication
(10) Pub. No.: US 2002/0077550 A1
(43) Pub. Date: Jun. 20, 2002

(54) Apparatus and method for treating gynecological diseases using an ultrasonic medical device operating in a transverse mode

(76) Inventors: Robert A. Rabiner, North Reading, MA (US); Bradley A. Hare, Chelmsford, MA (US)

Correspondence Address: PALMER & DODGE, LLP, RICHARD B. SMITH, 111 HUNTINGTON AVENUE, BOSTON, MA, 02199, US

(21) Appl. No.: 10/071,953

(22) Filed: Jan. 5, 2002

Related U.S. Application Data
(63) Continuation-in-part of application No. 09/975,725, filed on Oct. 11, 2001.
Application No. 09/975,725 is a continuation-in-part of application No. 09/625,803, filed on Jul. 26, 2000.
(60) Provisional application No. 60/157,824, filed on Oct. 5, 1999.
(60) Provisional application No. 60/225,060, filed on Aug. 14, 2000.

(51) Int. Cl. A61B 17/22

(52) U.S. Cl. 600/439; 600/411

(57) ABSTRACT

An apparatus and a method for treating gynecological diseases using an ultrasonic medical device operating in a transverse mode to cause fragmentation of diseased tissue without the excision of adjacent non-diseased tissue. The ultrasonic medical device includes an elongated probe having a proximal end, a distal end, and a longitudinal axis that can be inserted into a body lumen, and an ultrasonic generator for providing ultrasonic energy to the elongated probe for emission along the longitudinal axis of the elongated probe to the tissue adjacent the body lumen, wherein the ultrasonic energy creates a standing transverse wave in the elongated probe. A method includes inserting a member having a longitudinal axis into the body cavity, providing ultrasonic energy to the member and generating an area of cavitation along the longitudinal axis of the member, and sweeping the member over the surface of the body cavity to destroy the targeted cells.

### RELATED APPLICATIONS

This application continuation in part of U.S. application Ser. No. 09/975,725, filed on Oct. 11, 2001, entitled “Ultrasonic Probe With Rapid Attachment And Detachment Means,” which is a continuation in part of U.S. application Ser. No. 09/625,803, filed on Jul. 26, 2000, which claims priority to U.S. Provisional Application No. 60/157,824, filed on Oct. 5, 1999, and claims the benefit of U.S. Provisional Application No. 60/225,060, filed on Aug. 14, 2000, the entirety of these applications are hereby incorporated by reference.

### FIELD OF THE INVENTION

The present invention relates generally to medical devices, and particularly to an apparatus and a method for treating gynecological diseases using an ultrasonic medical device operating in a transverse mode to cause fragmentation of diseased tissue without the excision of adjacent non-diseased tissue. The present invention relates to an apparatus emitting ultrasonic energy in a transverse mode for treatment of gynecological diseases, and a method for treating gynecological diseases using ultrasonic energy.

### BACKGROUND OF THE INVENTION

Many gynecological procedures require removal of diseased tissue from the organs of the female reproductive system. For example, endometriosis is a condition in which fragments of the tissue lining the uterus (endometrium) spread to other tissues, such as the wall of the uterus, the ovaries, the peritoneum, or the bowel. The causes of endometriosis are unknown, but its incidence is higher in women who defer pregnancy until an advanced age. The fragments are benign, but may cause complications if they lodge in a critical location, leading to dysfunction of an organ. There are no definite symptoms of endometriosis, and the condition is usually found during a surgical operation for other disorders. Symptoms of endometriosis can include: heavy periods, often more frequent than usual, accompanied by pain (dysmenorrhea); pain during sexual intercourse (dyspareuria); infertility; and pain on defecation during a period. The abnormally placed fragments of endometrium pass through the same monthly cycle as does the normal endometrium-they grow thicker and swell before a menstrual period and then bleed. Because there is no outlet for the blood, cysts form. The cysts occasionally rupture, causing severe abdominal pain. In milder cases, painkilling drugs may lesson the symptoms. In severe cases, surgery or laser treatment are currently the only two options. As one of the severe cases, cul-de-sac obliteration implies the presence of retrocervical deep fibrotic endometriosis. The deep fibrotic endometriosis is usually located on the upper vagina, on the superficial anterior rectum, in the rectovaginal space, in the space between the upper vagina and the cervix (cervicovaginal angle), or in one or both uterosacral ligaments. With deep cul-de-sac obliteration, fibrotic endometriosis or adhesions sometimes involve the entire area between the cervicovaginal junction and the rectovaginal septum.

Other areas of the female reproductive system, for example, organs such as the ovaries and the uterus (especially the cervical region) are prone to developing malignant lesions. Lesions can also appear on vaginal and vulvar surfaces. A trend toward more conservative surgical intervention is evident in the current management of many gynecologic malignancies. Current medical management of vulvar carcinoma, for example, previously involved radical vulvectomy and bilateral lymphadenectomy, but current approaches consist of more limited excision of the primary tumor as well as of the regional lymph nodes. In preinvasive cervical carcinoma, conization is preferred instead of hysterectomy. The possibility for a more conservative surgical approach is also being explored for the treatment of selected early stage and advanced or recurrent cervical carcinomas. Although the primary surgical treatment of endometrial carcinoma remains unchanged, the necessity to universally perform more extensive procedures required for staging tumor development is being questioned. In early stage borderline ovarian tumors, not only adnexectomy but cystectomy alone is considered acceptable and re-exploration for staging purposes may not always be warranted. For example, in stage IA invasive carcinoma, adnexectomy of the involved side only is probably also sufficient. In advanced ovarian carcinoma, the more aggressive cytoreduction involving multiple organ resection is being questioned in view of more conservative approaches.

Loop Electrosurgical Excision Procedure (LEEP) or Large Loop Excision of the Transformation Zone (LLETZ) are special surgical techniques used for diagnosis and treatment of cervical dysplasia (pre-cancerous cells). LEEP or LLETZ can effectively cure the abnormal area on the cervix by surgically removing the abnormal cells. Prior to performing the LEEP, an examination of the cervix through a maginfiying device to detect abnormal cells (colposcopy) should be performed to rule out invasive cervical cancer and to determine the extent of the abnormal area of the cervix. During the LEEP, a gynecologist will use electrosurgery and a special thin wire loop to excise or remove the superficial layer of the cervix containing the abnormal cells. The extent of the area to be removed is determined by the extent of the area of abnormal cells as visualized by colposcopy. After the layer of abnormal cells is removed, the treated area is coagulated with a special ball electrode to help prevent bleeding. Silver nitrate, tannic acid solution, or ferric subsulfate, such as Monsel's solution, is then applied to the treated area and bleeding is thoroughly controlled.

Ablation therapy of the diseased tissue is effectuated by using cutting devices such as electrocautery devices or cryoprobes, which create extremes in temperature at the site of a lesion as a means of destroying the diseased cells. For example, electrocautery is used in gynecology to ablate the endometrial lining of the uterus. This procedure is often performed using a conductive roller similar in shape to a football which heats a wide swath of tissue along the inner surface of the uterus. Electrocautery has found broad general application in the treatment of endometriosis and in the removal of uterine fibroids and cancerous masses. The laser culdotomy, a procedure for the removal of cul-de-sac obliteration, poses a risk of unwanted tissue perforation or tissue vaporization. A less invasive approach using radio frequency therapeutic protocols, has been proven to be highly effective when used by electrophysiologists for the treatment of tachycardia; by neurosurgeons for the treatment of Parkinson's disease; and by neurosurgeons and anesthetists for other radio frequency procedures such as Gasserian ganglionectomy for trigeminal neuralgia and percutaneous cervical cordotomy for intractable pains. Radio frequency treatment, when coupled with a temperature control mechanism, can supply precise energy to the device-to-tissue contact site to obtain the desired thermal energy for treatment. But this method, and the methods of laser ablation and electrocautery, use thermal energy to destroy tissues.

At the other extreme, cryosurgery, the freezing and destruction of the diseased tissue, can also be used to treat gynecological diseases. In cryosurgery, a super cold probe (usually cooled by carbon dioxide, nitrogen, or similar gases) comes in contact with a focal point of the diseased tissue, freezing and destroying the diseased tissue by causing necrosis. Necrosis is the death of cells or tissues through injury or disease, especially in a localized area of the body. However, current methods of ablation therapy of the diseased tissue generally cause the necrosis of non-diseased tissue in the treatment area. The necrosis of otherwise healthy tissue in the region of the surgical procedure can complicate recovery and provide a potential site of post-operative infection. What is needed is a method and apparatus for performing gynecological surgical procedures that results in minimal tissue necrosis to the surrounding healthy tissues.

A secondary effect of the treatment of tissue, particularly in the areas of endometriosis and fibroid removal, is that separated tissue fragments typically remain in the working area and must be periodically flushed or suctioned away to preserve the required visibility necessary for surgery. The surgical procedure is thus slowed by this need to remove fragments which obstruct visibility. Therefore, the requirement for intermittent clearing of the surgical site prolongs the gynecological procedure. It is known that ultrasound can add significant value to tissue resection and ablation procedures. Using high-frequency ultrasound, anatomical landmarks and tissue features can be imaged in depth, which cannot be done by optical instruments. Depth information provides improved guidance and monitoring capabilities. It enables the surgeon to monitor the progress of tissue treatment, and thereby lessens the risk of complications. In addition, the improved visualization provided by ultrasound can help to shorten procedure times. For example, to perform ultrasound measurements during electrocautery, the surgical probes for the electrocautery procedure must first be removed and thereafter, ultrasound introduced. Finally, and after such measurements, surgery can resume with reintroduction of the surgical probes. With such procedures, the surgeon has difficulty returning to the original surgical site. For this reason, ultrasound is not usually utilized for measurement of uterine wall thickness by an intrauterine transducer. What is needed is an improved method of visualizing the site of a gynecological surgical procedure that does not disrupt the procedure.

Ultrasonic probes are devices which use ultrasonic energy to fragment body tissue (see, e.g., U.S. Pat. Nos. 5,112,300; 5,180,363; 4,989,583; 4,931,047; 4,922,902; and 3,805,787) and have been used in surgical procedures. The ultrasonic energy produced by an ultrasonic probe is in the form of very intense, high frequency sound vibrations that result in powerful chemical and physical reactions in the water molecules within a body tissue or surrounding fluids in proximity to the probe. These reactions ultimately result in a process called “cavitation,” which can be thought of as a form of cold (i.e., non-thermal) boiling of the water in the body tissue, such that microscopic bubbles are rapidly created and destroyed in the water, creating cavities in their wake. As surrounding water molecules rush in to fill the cavity created by the collapsed bubbles, they collide with each other with great force. The shock waves running outward from the collapsed bubbles can fragment or ablate material such as surrounding tissue in the vicinity of the probe.

Some ultrasonic probes include a mechanism for irrigating an area where the ultrasonic treatment is being performed (e.g., a body cavity or a lumen) to wash tissue debris from the area. Mechanisms used for irrigation or aspiration described in the prior art are generally structured such that they increase the overall cross-sectional profile of the probe, by including inner and outer concentric lumens within the probe to provide irrigation and aspiration channels for removal of particulate matter. In addition to making the probe more invasive, prior art probes also maintain a strict orientation of the aspiration and the irrigation mechanism, such that the inner and outer lumens for irrigation and aspiration remain in a fixed position relative to one another, which is generally closely adjacent to the area of treatment. Thus, the irrigation lumen does not extend beyond the suction lumen (i.e., there is no movement of the lumens relative to one another) and any aspiration is limited to picking up fluid and/or tissue remnants within the defined distance between the two lumens.

Another drawback of prior art ultrasonic medical probes is that they typically remove tissue relatively slowly in comparison to instruments that excise tissue by mechanical cutting. Part of the reason for this is that prior art ultrasonic devices rely on a longitudinal vibration of the tip of the probe for their tissue-disrupting effects. Because the tip of the probe is vibrated in a direction in line with the longitudinal axis of the probe, a tissue-destroying effect is only generated at the tip of the probe.

A solution that has been proposed is to vibrate the tip of the probe in a direction other than perpendicular to the longitudinal axis of the probe, in addition to vibrating the tip in the longitudinal direction. It is proposed that such motions will supplement the main point of tissue destruction, which is at the probe tip, since efficiency is determined by surface area of the probe tip. For example, U.S. Pat. No. 4,961,424 to Kubota, et al. discloses an ultrasonic treatment device that produces both a primary longitudinal motion, and a supplementary lateral motion of the probe tip to increase the tissue disrupting efficiency. The Kubota, et al. device, however, still relies primarily on the tip of the probe to act as a working surface. The ancillary lateral motion of the probe is intended to provide an incremental efficiency for the device operation. Thus, while destruction of tissue in proximity to the tip of the probe is more efficient, tissue destruction is still predominantly limited to the area in the immediate vicinity at the tip of the probe. Kubota, et al. disclosure is therefore limited in its ability to ablate tissue within inner surfaces of cylindrical blood vessels, for example, in vascular occlusions.

U.S. Pat. No. 4,504,264 to Kelman discloses an ultrasonic treatment device containing a probe that is capable of longitudinal vibrations and lateral oscillation. The Kelman patent is intended to improve the efficiency of ultrasonic tissue removal by providing a dual function of a fragmentation and a cutting device. Tissue fragmentation is caused primarily by oscillating the tip of the probe in addition to relying on longitudinal vibrations of the probe. Tissue fragmentation is caused primarily at the tip of the device, while the oscillatory motion can be employed by the surgeon to cut tissue, thereby increasing efficiency of surgical procedures. Additionally, the prior art requires complex instrument design and the incorporation of a plurality of electrodes, ultrasound frequency generating elements, switches and/or voltage controllers.

Accordingly, there is a need in the art for a medical device and method which remedies these limitations, for efficient fragmentation and removal of undesirable tissues implicated in gynecological diseases, particularly endometriosis and cancerous lesions or masses. In particular, there is a need in the art for methods and devices for enhancing the performance of procedures involving the ablation of diseased tissues from a healthy organ. There is also a need in the art for mechanisms and methods that decrease the creation of necrotic tissue and minimize local tissue damage, and that can be used concurrently with imaging systems for greater precision.

### SUMMARY OF THE INVENTION

The present invention is an apparatus and a method for treating gynecological diseases using an ultrasonic medical device operating in a transverse mode to cause fragmentation of diseased tissue without the excision of adjacent non-diseased tissue. The ultrasonic medical device includes an elongated probe having a proximal end, a distal end, and a longitudinal axis wherein the elongated probe can be inserted into a body lumen, and an ultrasonic generator for providing ultrasonic energy to the elongated probe for emission along the longitudinal axis of the elongated probe to the tissue adjacent the body lumen, wherein the ultrasonic energy creates a standing transverse wave in the elongated probe such that a plurality of nodes and a plurality of anti-nodes are formed along the longitudinal axis of the elongated probe to treat the tissue adjacent the body lumen. A method for treating gynecological diseases by destroying targeted cells on a surface of a body cavity includes inserting a member having a longitudinal axis into the body cavity, providing ultrasonic energy to the member and generating an area of cavitation along the longitudinal axis of the member, and sweeping the member over the surface of the body cavity to destroy the targeted cells.

In general, it is an object of the present invention to provide a method and a medical device to treat by ablation, such diseases as endometriosis, cysts, polyps, tumors, or abnormal cellular growths or lesions of tissues of the female reproductive tract and surrounding areas. The present invention has particular application in removal of abnormal cell growths on the surface of the tissues of the female reproductive system, or organs such as the uterus, cervix or ovaries, including, but not limited to, leiomyoma (fibroids) such as intramural, subserous, and submucous leiomyoma, invasive epidermoid carcinoma, cervical intraepithelial neoplasia (CIN) grades 1, 2, and 3, adnexal mass of the fallopian tubes, ovarian tumors such as epithelial tumors, gonadal stromal tumors, and germ cell tumors, endometrial polyps, endometrial hyperplasia, adenomateous hyperplasia of the endometrium, and carcinoma in situ of the endometrium. In one embodiment of the present invention, the target tissue is cervical or uterine tissue, and the tissue destroyed comprises an abnormal cell growth. In another embodiment of the present invention, the abnormal cell growth is fibroid growth or polyp. In a further embodiment of the present invention, the abnormal cell growth is a cancer mass or cancerous lesion. In another embodiment of the present invention, the ultrasonic medical device is used to treat endometriosis.

The ultrasonic medical device according to the present invention emits transverse ultrasonic energy along the length of the probe, fragmenting abnormal cells on the surface of the body cavity, which come within the sweep of the probe. The ultrasonic medical device is designed to have a small cross-sectional profile, allowing it to be used in a minimally-invasive manner. The ultrasonic medical device according to the present invention has a cross-sectional profile of less than about 3 millimeters (approximately the size of a cervix). Thus, there is not a need to significantly dilate the cervix to use the present invention to ablate diseased tissue of the cervix. Dilating the cervix causes patient discomfort and the present invention's ability to ablate diseased tissue of the cervix without dilating the cervix results in a more comfortable procedure for the patient. The less than about 3 millimeter cross-sectional profile of the present invention includes any irrigation channels and aspiration channels if they are present.

In several embodiments of the present invention, the ultrasonic medical device is covered with an acoustic sheath, such as the sheaths described in the Assignee's co-pending patent application U.S. Ser. No. 09/784,619, which is hereby incorporated by reference, to modulate transversely emitted energy, and deliver it in a precise manner to specific target tissue sites. In one embodiment of the present invention, the probe is used with an aspiration sheath to enhance the ability of the probe to remove fluids and tissue debris from a surgical site, and an irrigation sheath to deliver irrigation fluids and pharmaceutical agents to the site. In a further embodiment of the present invention, the sheath has the ability to manipulate sections of tissue in conjunction with the actions of the ultrasonic probe. In another embodiment of the present invention, the sheaths are used in conjunction with the probe and an ultrasonic imaging system. Each of the sheaths provides optional functionalities which the user (e.g., a physician, a nurse, or a technician) can select from to suit their needs.

In one embodiment of the present invention, the ultrasonic medical device, that may also contain a sheath, is supplied in a container. The container maintains the sterility of a probe and provides a method for attaching the probe to the transducer of an ultrasonic medical device without direct handling of the probe. The container also provides a method for housing a used probe, which would be considered medical waste, thereby enabling disposal of the probe in compliance with safety regulations governing the disposal of sharp medical instruments. In another embodiment of the present invention, the container provides a method for removal of a contaminated probe from the transducer of an ultrasonic medical device. Such containers for use with probes are described in the Assignee's co-pending patent application U.S. Ser. No. 09/975,725, which is hereby incorporated by reference.

In another embodiment of the present invention, the ultrasonic medical device is used with an imaging system, such as ultrasound, magnetic resonance imaging, an endoscope, or a laproscope.

In another embodiment, the present invention provides for a kit including an ultrasonic medical device and instructions for its use in a gynecological procedure, i.e., instructions for power settings and frequency settings for a particular procedure and for a particular probe shape and size. Other useful items for inclusion in such a kit are one or more sheaths that can be adapted to the probe. In a further embodiment of the present invention, the kit further comprises a sharps container. In the preferred embodiment of the present invention, the items in the kit are sterilized and pre-assembled, and the kit is hermetically sealed against environmental and microbial contaminants.

### BRIEF DESCRIPTION OF THE DRAWINGS

The present invention will be further explained with reference to the attached drawings, wherein like structures are referred to by like numerals throughout the several views. The drawings shown are not necessarily to scale, with emphasis instead generally being placed upon illustrating the principles of the present invention.

FIG. 1A is a side plan view of an ultrasonic medical device operating in a transverse mode of the present invention for treatment of gynecological diseases.

FIG. 1B is a side plan view of an ultrasonic medical device operating in a transverse mode of the present invention showing a plurality of nodes and a plurality of anti-nodes along an active area of a transverse mode probe.

FIG. 2 is a perspective view of a transverse mode probe of the present invention including a sheath.

FIG. 3A is a perspective view of a transverse mode probe of the present invention including a first sheath and a second sheath.

FIG. 3B is a perspective view of a transverse mode probe of the present invention showing a sheath in relation to a probe.

FIG. 3C is a perspective view of a transverse mode probe of the present invention showing a sheath with a plurality of fenestrations.

FIG. 3D is a perspective view of a transverse mode probe of the present invention showing a sheath with a plurality of fenestrations and reflective elements.

FIG. 3E is a perspective view of a transverse mode probe of the present invention showing a sheath comprising two semi-cylindrical sections where a first semi-cylindrical section connected to a second semi-cylindrical section by at least one connecting means.

FIG. 3F is a perspective view of a transverse mode probe of the present invention showing a sheath comprising at least two cylindrical sections where a first cylindrical section connected to a second cylindrical section by at least one connecting means.

FIG. 4 is an enlarged perspective view of a tip of a transverse mode probe of the present invention.

FIG. 5 is a perspective view of an articulable transverse mode probe of the present invention.

FIG. 6 is a longitudinal sectional view of a portion of a transverse mode probe of the present invention showing a central irrigation passage, lateral irrigation lumens, and external a aspiration channels.

FIG. 7 is a transverse sectional view of a portion of a transverse mode probe of the present invention as shown in FIG. 6.

FIG. 8 is a perspective view of a transverse mode probe of the present invention showing an aspiration sheath and a rigid outer sheath.

FIG. 9 is a perspective view of a transverse mode probe of the present invention showing a sheath or catheter with a grasping element according to one embodiment of the present invention.

FIG. 10 is a perspective view of a transverse mode probe of the present invention showing an aspiration housing for connecting the transverse mode probe to a source of negative pressure.

FIG. 11A is an enlarged fragmentary view of a reflective element, including a plurality of planar surfaces, for use in a sheath.

FIG. 11B is an enlarged fragmentary view of a reflective element, including a plurality of curved surfaces, for use in a sheath.

FIG. 11C is a sectional view of a transverse mode probe of the present invention at least partially covered by a sheath having at least one reflective element.

FIG. 12 is an enlarged fragmentary view of a transverse mode probe of the present invention including a sheath, an optical imaging element, an image data transmitter, and a display.

While the above-identified drawings set forth preferred embodiments of the present invention, other embodiments of the present invention are also contemplated, as noted in the discussion. This disclosure presents illustrative embodiments of the present invention by way of representation and not limitation. Numerous other modifications and embodiments can be devised by those skilled in the art which fall within the scope and sprit of the principles of the present invention.

### DETAILED DESCRIPTION

The present invention is an apparatus and a method for treating gynecological diseases using an ultrasonic medical device operating in a transverse mode to cause fragmentation of diseased tissue without the excision of adjacent non-diseased tissue. The present invention provides an ultrasonic medical device operating in a transverse mode for gynecological applications where ablation of diseased tissue is desired. By physically destroying and removing tissue through ultrasonic energy, a non-thermal method, surgical complications are reduced, and patient pain and discomfort is minimized.

The following terms and definitions are used herein:

“Cavitation” as used herein refers to shock waves produced by ultrasonic vibration, wherein the vibration creates a plurality of microscopic bubbles which rapidly collapse, resulting in molecular collision by water molecules which collide with force thereby producing the shock waves.

“Fenestration” as used herein refers to an aperture, window, opening, hole, or space.

“Node” as used herein refers to a region of minimum energy emitted by an ultrasonic probe at or proximal to a specific location along the longitudinal axis probe.

“Anti-node” as used herein refers to a region of maximum energy emitted by an ultrasonic probe at or proximal to a specific location along the longitudinal axis probe.

“Probe” as used herein refers to a device capable of being adapted to an ultrasonic generator means, which is capable of propagating the energy emitted by the ultrasonic generator means along its length, resolving this energy into effective cavitational energy at a specific resonance (defined by a plurality of nodes and a plurality of anti-nodes at pre-determined locations along an “active area” of the probe) and is capable of acoustic impedance transformation of ultrasound energy to mechanical energy.

“Sheath” as used herein refers to a device for covering, encasing, or shielding in whole or in part, a probe or portion thereof connected to an ultrasonic generation means.

“Transverse” as used herein refers to vibration of a probe at right angles to the axis of a probe. A “transverse wave” as used herein is a wave propagated along an ultrasonic probe in which the direction of the disturbance at each point of the medium is perpendicular to the wave vector.

“Tuning” as used herein refers to a process of adjusting the frequency of the ultrasonic generator means to select a frequency that establishes a standing wave along the length of the probe.

The present invention provides an ultrasonic medical device operating in a transverse mode for removing diseased tissues from the female reproductive tract by causing fragmentation of such diseased tissue without the excision of adjacent non-diseased tissue. Because the ultrasonic medical device is minimally invasive, flexible and articulable, it can be inserted into narrow, tissue spaces in the vagina and abdomen without risking damage to surrounding healthy tissues and organs, or blood vessels that supply these tissues and organs. Transverse vibration of the probe of an ultrasonic medical device generates a plurality of nodes and a plurality of anti-nodes of cavitation energy along the longitudinal axis of the probe, which are resolved into a plurality of cavitational anti-nodes emanating radially from the nodes and anti-nodes at specific points along the active portion of the probe. The target tissue is fragmented to debris approximately of sub-micron sizes, and the transverse vibration generates a retrograde flow of debris that carries the debris away from the probe tip.

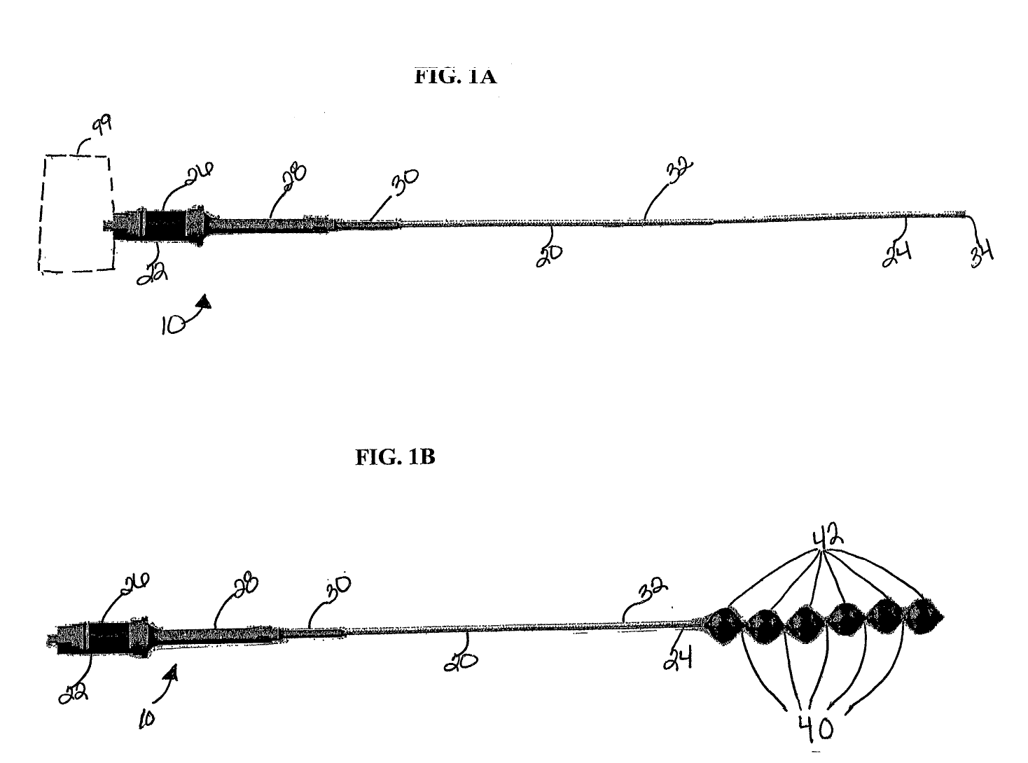

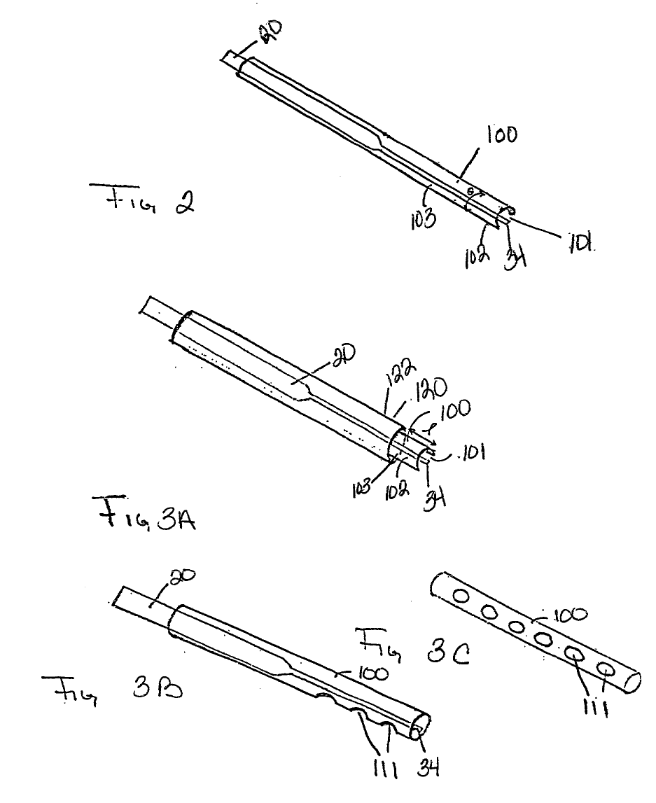

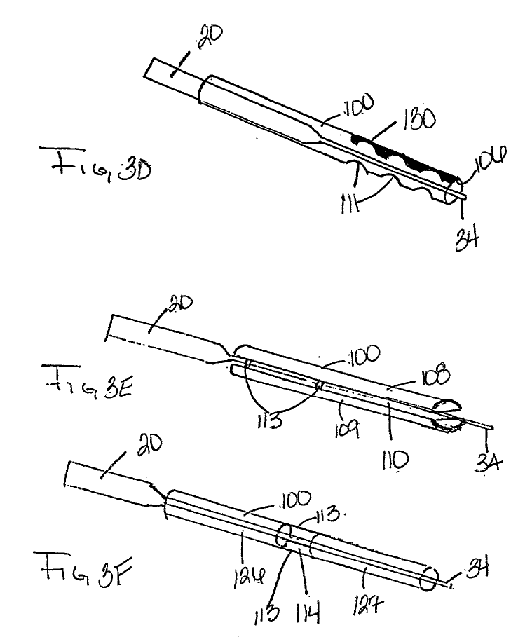

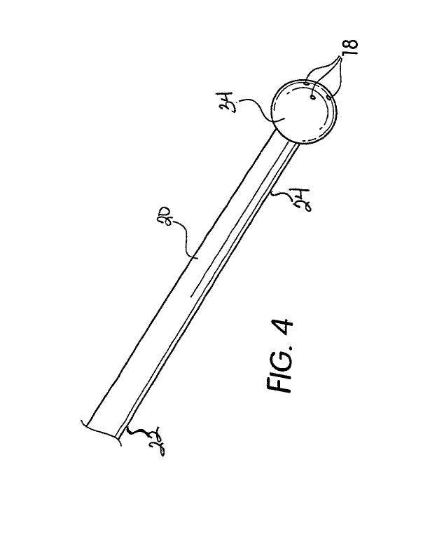

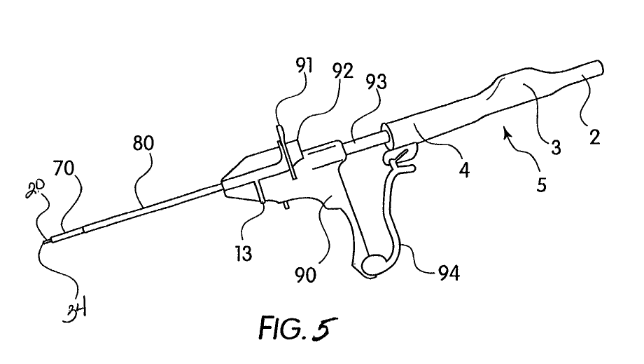

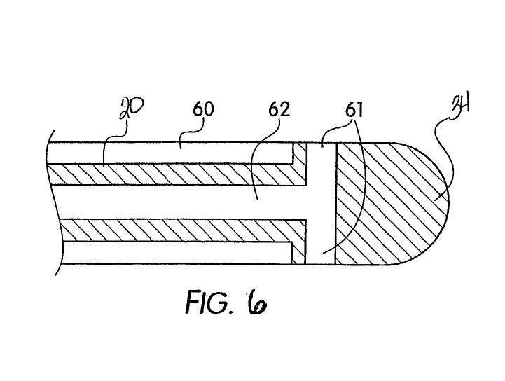

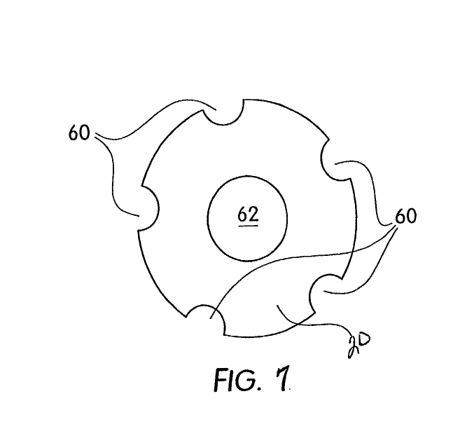

An ultrasonic medical device operating in a transverse mode of the present invention is illustrated generally at 10 in FIG. 1A. The ultrasonic medical device operating in a transverse mode includes an elongated probe 20 which is coupled to a device providing a source or a generator 99 (shown in phantom in FIG. 1A) for the production of ultrasonic energy. The ultrasonic generator 99 may or may not be a physical part of the ultrasonic medical device of the present invention itself. The probe 20 transmits ultrasonic energy received from the generator 99 along the length the probe 20. The probe 20 includes a proximal end 22 and a distal end 24. The probe 20 is capable of engaging the ultrasonic generator 99 at the proximal end 22 with sufficient restraint to form an acoustical mass that can propagate the ultrasonic energy provided by the ultrasonic generator 99. The distal end 24 of the probe 20 is a thin terminal interval ending in a probe tip 34, which has a small diameter enabling the distal end 24 to flex longitudinally. The probe tip 34 can be any shape including, but not limited to, bent so that the probe tip 34 is not just longitudinal, or bigger shapes for removing a larger area of tissue. In one embodiment of the present invention shown in FIG. 1A, a diameter of the probe 20 decreases at defined intervals 26, 28, 30, and 32. Energy from the ultrasonic generator 99 is transmitted along the length of the probe 20, causing the probe 20 and the probe tip 34 to vibrate.

The transverse mode of vibration of the ultrasonic probe according to the present invention differs from the axial (or longitudinal) mode of vibration disclosed in the prior art. Rather than vibrating in the axial direction, the probe vibrates in a direction transverse (perpendicular) to the axial direction. As a consequence of the transverse vibration of the probe 20, the tissue-destroying effects of the device are not limited to those regions of a tissue coming into contact with the probe tip 34. Rather, as the active portion of the probe 20 is positioned in proximity to a diseased area or lesion, the tissue is removed in all areas adjacent to the multiplicity of energetic anti-nodes that are produced along the entire length of the probe 20, typically in a region having a radius of up to about 6 mm around the probe 20.

Transversely vibrating ultrasonic probes for tissue ablation are described in the Assignee's co-pending patent applications (U.S. Serial Nos. 09/766,015, 60/178,901 and 60/225,060) which further describe the design parameters for such a probe and its use in ultrasonic devices for tissue ablation and the entirety of these applications are hereby incorporated by reference.

As a consequence of the probe design, the ultrasonic energy propagates along the length of the probe 20, and along the probe terminal interval 32 the ultrasonic energy manifests as a series of transverse vibrations, rather than longitudinal vibrations. As shown in FIG. 1B, a plurality of nodes 40 occur along the length of the probe 20 and at the probe tip 34 at repeating intervals. The nodes 40 are areas of minimum energy and minimum vibration. A plurality of anti-nodes 42, or areas of maximum energy and maximum vibration, also occur at repeating intervals along the probe 20 and at the probe tip 34. The number of nodes 40 and anti-nodes 42, and their spacing along the probe 20 depends on the frequency of the energy produced by the ultrasonic generator 99. The separation of the nodes 40 and the anti-nodes 42 is a function of harmonic intervals of the frequency, and can be affected by tuning the probe 20. In a properly tuned probe 20, the anti-nodes 42 will be found at a position exactly one-half of the distance between the nodes 40 located adjacent each side of the anti-node 42. The tissue-destroying effects of the ultrasonic medical device operating in a transverse mode of the present invention 10 are not limited to those regions of a tissue coming into contact with the probe tip 34. Rather, as the probe 20 is swept through an area of the tissue, preferably in a windshield-wiper fashion, the tissue is removed in all areas adjacent to the plurality of anti-nodes 42 being produced along the entire length of the probe 20. The extent of the cavitation energy produced by the probe tip 34 is such that it extends outward from the probe tip 34 at the anti-nodes 42 for about 1-6 millimeters. In this way, actual treatment time using the transverse mode ultrasonic medical device according to the present invention 10 is greatly reduced as compared to methods disclosed in the prior art.

By eliminating the axial motion of the probe and allowing transverse vibrations only, the active probe can cause fragmentation of large areas of tissue spanning the entire length of the active portion of the probe due to generation of multiple cavitational anti-nodes along the probe length perpendicular to the probe axis. Since substantially larger affected areas can be denuded of the diseased tissue in a short time, actual treatment time using the transverse mode ultrasonic medical device according to the present invention is greatly reduced as compared to methods using prior art probes that primarily utilize longitudinal vibration (along the probe axis) for tissue ablation. A distinguishing feature of the present invention is the ability to utilize probes of extremely small diameter (about 0.025 inches and smaller) compared to prior art probes without loss of efficiency, because the tissue fragmentation process in not dependent on the area of the probe tip (distal end). Highly flexible probes can therefore be designed to mimic device shapes that enable facile insertion into tissue spaces or extremely narrow interstices. Another advantage provided by the present invention is the ability to rapidly remove tissue from large areas within cylindrical or tubular surfaces such as the fallopian tubes or selected areas within and along the walls of the uterus, which is not possible by prior art devices that rely on the longitudinal vibrating probe tip for effecting tissue fragmentation.

A significant advantage of the present invention is that it physically destroys and removes adipose or other high water content tissue through the mechanism of non-thermal cavitation, which makes it well suited for use in treating gynecological diseases. The removal of tissue by cavitation also provides the ability to remove large volumes of tissue with a small diameter probe, without making large holes in the tissue or the surrounding areas. Accordingly, because of the use of cavitation as the mechanism for destroying tissue, together with the use of irrigation and aspiration, the method and apparatus of the present invention can destroy and remove tissue within a range of temperatures of ±7° C. from normal body temperature. Therefore, complications attendant with the use of thermal destruction or necrosis of tissue—such as swelling or edema, as well as loss of elasticity are avoided. Furthermore, the use of fluid irrigation can enhance the cavitation effect on surrounding tissue, thus speeding tissue removal.

The cavitation energy is the energy that is expelled from the probe in a stream of bubbles which must contact the tissue to cause ablation. Therefore, blocking the cavitation bubble stream from contacting tissue will spare the tissue from ablation, while directing the cavitation bubble stream to contact the tissue will cause ablation.

The number of nodes 40 and anti-nodes 42 occurring along the axial length of the probe is modulated by changing the frequency of energy supplied by the ultrasonic generator 99. The exact frequency, however, is not critical and the ultrasonic generator 99 run at, for example, 20 kHz is generally sufficient to create an effective number of tissue destroying anti-nodes 42 along the axial length of the probe. In addition, as will be appreciated by those skilled in the art, it is possible to adjust the dimensions of the probe 20, including diameter, length, and distance to the ultrasonic energy generator 99, in order to affect the number and spacing of the nodes 40 and anti-nodes 42 along the probe 20. The present invention allows the use of ultrasonic energy to be applied to tissue selectively, because the probe 20 conducts energy across a frequency range of from about 20 kHz through about 80 kHz. The amount of ultrasonic energy to be applied to a particular treatment site is a function of the amplitude and frequency of vibration of the probe. In general, the amplitude or throw rate of the energy is in the range of 150 microns to 250 microns, and the frequency in the range of about 20,000 Hertz to about 80,000 Hertz (20 kHz 80 kHz). In a preferred embodiment of the present invention, the frequency of ultrasonic energy is from about 20,000 Hertz to about 35,000 Hertz (20 kHz-35 kHz). Frequencies in this range are specifically destructive of hydrated (water-laden) tissues such as endothelial tissues, while substantially ineffective toward high-collagen connective tissue, or other fibrous tissues including, but not limited to, vascular tissues, epidermal, or muscle tissues.

In a preferred embodiment of the present invention, the ultrasonic generator 99 is mechanically coupled to the proximal end 22 of the probe 20 to oscillate the probe 20 in a direction transverse to its longitudinal axis. Alternatively, a magneto-strictive generator may be used for generation of ultrasonic energy. The preferred generator is a piezoelectric transducer that is mechanically coupled to the probe 20 to enable transfer of ultrasonic excitation energy and cause the probe 20 to oscillate in a transverse direction relative to its longitudinal axis. The ultrasonic medical device 10 is designed to have a small cross-sectional profile, which also allows the probe 20 to flex along its length, thereby allowing the probe 20 to be used in a minimally invasive manner. Transverse oscillation of the probe 20 generates a plurality of cavitation anti-nodes 42 along the longitudinal axis of the probe 20, thereby efficiently destroying the tissues that come into proximity with the energetic anti-nodes 42. A significant feature of the present invention resulting from the transversely generated energy is the retrograde movement of debris, e.g., away from the probe tip 34 and along the shaft of the probe 20.

The amount of cavitation energy to be applied to a particular site requiring treatment is a function of the amplitude and frequency of vibration of the probe 20, as well as the longitudinal length of the probe 20, the proximity of the probe 20 to a tissue, and the degree to which the probe 20 length is exposed to the tissue. The length of the probe 20 that is exposed to the tissue can be controlled by utilizing a sheath 100 with the probe 20 to contain the emitted energy and focus and deliver the emitted energy to the desired location.

FIG. 2 shows a transverse mode probe 20 according to one embodiment of the present invention where a sheath 100 partially contains the distal end 24 of the probe 20. For purposes of illustration, the probe 20 is visible beneath the sheath 100. The semi-cylindrical sheath 100 has a semi-rigid wall 103 that surrounds a portion of probe 20 and extends longitudinally along the probe 20. A terminal end 102 of the sheath 100 has an opening 101 also extending longitudinally along the probe 20, the opening 101 provides a window for directing cavitation energy generated by the probe 20, i.e., the stream of cavitation bubbles, toward the tissue to be destroyed. Conversely, the semi-rigid wall 103 blocks cavitation energy generated by the probe 20 from reaching the tissue on the opposite side of the wall, i.e., the tissue to be spared. The probe 20 is substantially contained within the semi-cylindrical sheath 100 that is capable of modulating the energy omitted by an active probe 20, and shielding tissues from puncture by the probe tip 34. The sheath 100 is semi-cylindrical and of a sufficient diameter to at least partially encompass the probe 20. The angular extent θ of the sheath may vary depending on the tissue removal requirements, and will generally extend from less than about 180 degrees to more than about 270 degrees. In the semi-cylindrical embodiment shown in FIG. 2, a circumference of the sheath 100 is approximately 180 degrees, and a length of the sheath 100 is sufficient to cover the plurality of intervals 30 and 32 of the probe 20.

The present invention allows the use of a plurality of sheaths 100 (with or without fenestrations). The sheath 100 can help the user gage the amount of tissue to be effected by moving the sheath 100 back and forth along the probe 20. The probe 20 itself has a small diameter with limited supportive strength. In one embodiment of the present invention, the smaller the probe diameter, the more effective the tissue ablation. However, the smaller the probe diameter, the less supportive strength is provided by the probe itself. Thus, the sheath 100 can provide support to the probe 20 to irrigate, aspirate, and cut tissue in a cold fashion. The sheath 100 can also be used a guide for the probe 20, giving the probe 20 support and making a more rigid ultrasonic medical device operating in a transverse mode of the present invention.

FIGS. 3A-F show additional configurations of the sheaths 100 for ultrasonic medical device 10 according to embodiments of the present invention. FIG. 3A shows a transverse mode probe 20 according to one embodiment of the present invention including the first sheath 100 that is semi-cylindrical (also shown in FIG. 2), and a second concentric sheath 120. In this embodiment of the present invention, the second sheath 120 is cylindrical, and is capable of containing the first sheath 100, as well as the probe 20. The probe 20 is substantially contained within the second cylindrical sheath 120. As shown in FIG. 3A, the terminal end 102 of the first sheath 100 has the opening 101 which exposes the probe 20 and the probe tip 34. A terminal end 122 of the second sheath 120 is shaped to provide a means for manipulating tissue to bring it into proximity with the probe tip 34. The second cylindrical sheath 120 surrounds a portion of the first sheath 100, and can be manipulated longitudinally along the first sheath 100 to provide a means for modulating the exposure of the probe 20 and the probe tip 34, and thereby modulating the cavitation energy emitted by the probe 20 to which the tissues will be exposed.

Referring to FIGS. 3B and 3C, the invention further provides a cylindrical sheath 100 shown with one or more fenestrations 111 along its length. The fenestrations 111 are apertures and can be round, square, or oval, in shape and provide a focused area of treatment where the intensity of the cavitation energy can be enhanced by the function of the sheath 100. That is, the cavitation energy emitted from the probe 20 will only pass though the fenestrations 111 to ablate adjacent tissue, and be blocked by the remainder of the sheath 100 to spare other tissue. The fenestrations 111 can be spaced and shaped such that they are capable of defining the shape and space associated with the stream of cavitation bubbles. FIG. 3B shows an embodiment of the present invention wherein the sheath 100 comprises a cylindrical structure of a sufficient diameter to contain the probe 20 which is visible for the purpose of illustration. The probe 20 is substantially contained within the sheath 100. The cavitation energy emitted by the probe 20 is constrained by the sheath 100 and is communicated to areas outside of the sheath 100 through the fenestrations 111. FIG. 3C is a view of only the sheath 100 that is hollow, cylindrical and contains a plurality of arcuate fenestrations 111.

FIG. 3D shows an embodiment of the present invention wherein the probe 20 is contained within the sheath 100 which includes a plurality of fenestrations 111, and at least one acoustic reflective element 130, which is adapted to an interior surface 106 of the sheath 100. The reflective elements 130 engage the interior surface 106 of the sheath 100 opposite the fenestrations 111. The reflective elements 130 focus the cavitation energy emitted from the probe 20 through the fenestrations 111 to enhance the intensity of the cavitation energy emitted through the fenestrations 111 so as to focus and intensify the energy directed towards to the treatment area. In a preferred embodiment, the reflective elements 130 are acoustic lenses.

FIG. 3E shows an embodiment of the present invention wherein the sheath 100 further includes two parallel semi-cylindrical sections 108, 109 extending along the length of probe 20 that are spaced apart in a sandwich-like fashion to form openings 110 along opposite sides of the probe 20. The semi-cylindrical section 108 is connected to the semi-cylindrical section 109 by one or more connecting means 113. The probe 20 is capable of being substantially contained within the connected semi-cylindrical sections 108, 109 of the sheath 100. The cavitation energy generated by the probe 20 is contained by the connected semi-cylindrical sections 108, 109, where the semi-cylindrical sections 108, 109 occlude the probe 20. The cavitation energy emitted from the probe 20 will only pass though openings 110 to ablate adjacent tissue, and be blocked by the remainder of the sheath 100 to spare other tissue. The embodiment shown in FIG. 3E will emit cavitation energy substantially along a plane coincident with the long axis of the probe 20, allowing formation of a flat, fan-like pattern.

FIG. 3F shows an embodiment of the present invention wherein the sheath 100 further includes at least two cylindrical sections 126, 127 extending along the length of probe 20 and separated by an annular aperture 114. The cylindrical section 126 is connected to the cylindrical section 127 by one or more connecting means 113. The probe 20 is capable of being substantially contained within the cylindrical sections 126, 127 of the sheath 100. The cavitation energy generated by the probe 20 is contained by the cylindrical sections 126, 127 of the sheath 100, where the cylindrical sections 126, 127 occlude the probe 20. The cavitation energy emitted from the probe 20 will only pass though the annular aperture 114 to ablate adjacent tissue, and be blocked by the remainder of the sheath 100 to spare other tissue. The embodiment shown in FIG. 3F will emit cavitation energy substantially radially along a plane perpendicular to the long axis of the probe 20, allowing formation of a disk pattern. The connecting means 113 (shown in FIG. 3E and FIG. 3F) can be any means of connecting two sections known in the art, including, but not limited to, adhesives, welding, coupling, clamping, fastening, and the like.

In another preferred embodiment of the present invention, the elongated probe of the present invention is circumferentially enclosed in a sheath that provides a conduit for irrigation fluids, aspiration of fragmented tissue, or for delivery of therapeutic drugs to the treatment site. The sheath can extend either partially or over the entirety of the probe, and can additionally comprise fenestrations for directing ultrasonic energy from the probe at specific locations within venal cavities for selective ablation of tissue. An ultrasonic tissue ablation device comprising a sheath for removal of occlusions in blood vessels has been disclosed in the Assignee's co-pending patent application U.S. Ser. No. 09/776,015, the entirety of which is hereby incorporated by reference.

In another preferred embodiment of the present invention, modulation of the probe energy, and as such, control over the tissue area exposed to such energy is effected by utilization of a probe sheath assembly. The sheath assembly comprises either a single sheath or multiple sheaths configured concentrically around the probe, enabling control of probe exposure to the surgical site. Sheath materials useful for the present invention include any material with acoustical or vibrational dampening properties capable of absorbing, containing, or dissipating the cavitation energy emitted by the probe tip. Such materials must be capable of being sterilized by, for example, gamma irradiation or ethylene oxide gas (ETO), without losing their structural integrity. Such materials include, but are not limited to, plastics such as polytetrafluoroethylene (PTFE), polyethylene, polypropylene, silicone, polyetherimide, or other plastics that those skilled in the art know are commonly used in medical devices. Ceramic materials can also be used, and have the added benefit of being able to be sterilized by heat and pressure, such as in an autoclave. Combinations of the aforementioned materials can be used depending on the procedure. For example, as best shown in FIG. 3A, the first sheath 100 may be composed of a ceramic, and can be used in combination with a moveable second outer sheath 120 that is composed of PTFE. Alternatively, a single sheath may employ two or more materials to give the desired combination of strength and flexibility. For example, the sheath 100 may comprise a rigid ceramic section distal to the probe tip 34 and a more flexible plastic section proximal to the probe tip 34, capable of flexing with the probe 20. In the currently preferred embodiment of the present invention, PTFE is used to fabricate a strong, flexible, disposable to sheath that is easily sterilized by irradiation or ETO gas.

The length and diameter of the sheath used in a particular surgical procedure will depend on the type of the probe selected, the extent and length to which the probe will be inserted into the body cavity or lumen, and the degree of shielding that is required. For example, the ultrasonic probe of the present invention may be used in a procedure to destroy diseased tissue from the interior of the uterus, a location deep inside a patient's body. For such a procedure, the sheath must be of a sufficient length to protect the vaginal and cervical tissues from the surgical insertion point to the site of the procedure, of a sufficient outer diameter to facilitate insertion of the sheath into the vessel, and of a sufficient inner diameter to enable acceptance of the probe. By contrast, for ablating, for example, vulvar lesions, the appropriate probe is one that is significantly shorter and as such, so would be the sheath. The exact length and diameter of the sheath 100 will be determined by the requirements of the surgical procedure. Similarly, the position and size of the sheath aperture or number and positions of the fenestrations, or the addition of a bevel on the sheath terminus, will likewise be determined by the type of procedure and the specific patient requirements.

The sheaths described may be used to cover the probe during treatment performed in delicate areas, such as is on the surface of the uterus or the cervix. The apertures in the sheath can further be covered with an outer cylindrical sheath described above. All the above described sheaths can be introduced and controlled with known techniques such as attaching the sheaths to, e.g., a guide-wire. Use of a larger diameter sheath can protect tissue from accidental penetration by an ultrasonic probe that may be stiffer than the surrounding tissue.

The sheath can be of fixed size and the sizes may vary depending on the size of the target tissue to be removed and the length of the probe. The size of the apertures of the sheath can also vary depending on the amount of cavitation energy that is desired to be directed to the target tissue.

A particular advantage of the ultrasonic probe operating in transverse mode is that the efficient cavitation energy produced by the probe disintegrates target tissues into sub-micron particles. The modality of operation of the probe propels tissue debris generated along the active area of the probe in a retrograde direction, away from the tip. Accordingly, a preferred embodiment of the present invention provides at least one aspiration channel that can be adapted to a vacuum or suction device, to remove the tissue debris created by the action of the probe. The aspiration channel can be fabricated from the same material as the sheath, provided it is of sufficient rigidity to maintain structural integrity under the negative pressure produced by the aspiration means. Such an aspiration channel could be provided inside the lumen of the sheath, or along the exterior surface of the sheath. Alternatively, the sheath itself may provide the aspiration channel.

A distinguishing feature of the present invention is the ability to utilize probes of extremely small diameter (narrow diameter probes) compared to prior art devices (large diameter probes) without loss of efficiency or efficacy, since the tissue fragmentation process is not dependent on the area of the probe tip (distal end). Highly flexible probes can therefore be obtained to mimic device shapes that enable facile insertion into highly occluded or extremely narrow interstices without resulting in breakage of the probe or puncture or damage of the tissue or body cavity while ensuring optimal results.

A second distinguishing feature of the small diameter probes of the present invention is that the probe diameter is approximately the same over their entire length, that is,—the active tip segment (distal end) and the rear segment (proximal end) of the probes are approximately similar in diameter. In a preferred embodiment of the present invention, the probe diameters at the proximal and distal ends respectively are about 0.025 inches. An advantage of the shape configuration of the probes of the present invention is that they are adaptable to currently used standard vascular introducers. Because the rear segment (proximal end) of the probes do not have a non-cylindrical shape or “bulk”, catheters and guides can be introduced over the ends of the elongated wire probes of the present invention, thereby—allowing their use in standard-configuration endovascular procedures.

The elongated probe of the present invention is either a single diameter wire with a uniform cross section offering flexural stiffness along its entire length, or is tapered or stepped along its length to control the amplitude of the transverse wave along its entire longitudinal axis. Alternatively, the probe can be cross-sectionally non-cylindrical and capable of providing both flexural stiffness and support energy conversion along its entire length. The length of the elongated probe of the present invention is chosen so as to be resonant in either in an exclusively transverse mode, or be resonant in combination of transverse and longitudinal modes to provide a wider operating range. In a preferred embodiment of the present invention, the elongated probe is chosen to be from about 30 centimeters to about 300 centimeters in length. In a most preferred embodiment of the present invention, the elongated probe has a length of about 70 centimeters to about 210 centimeters in length. Suitable probe materials include metallic materials and metallic alloys suited for ultrasound energy transmission. In a preferred embodiment of the present invention, the metallic material comprising the elongated probe is titanium.

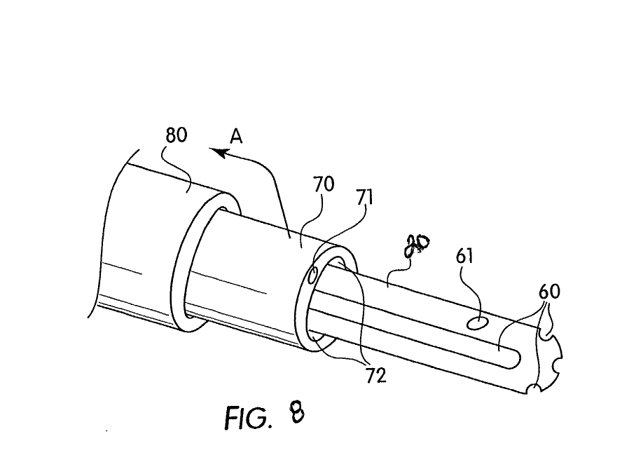

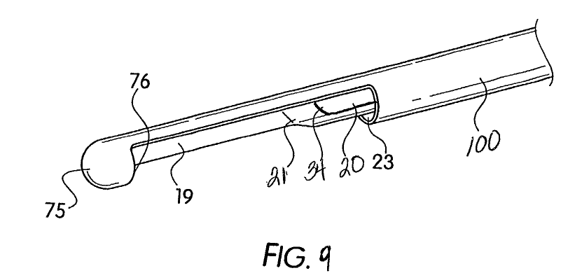

FIG. 4 is an enlarged perspective view of the probe tip 34 of a transverse mode probe 20 of the present invention. The body of the ultrasonic probe 20 in the embodiment of FIG. 4 is preferably slightly tapered from the proximal end 22 to the distal end 24. The probe tip 34 is in the form of a ball-shaped projection from the end of the probe 20. This shape of the probe tip 34 eliminates any sharp edges or surfaces on the probe tip 34 which could result in damage to tissue during insertion, treatment or removal. The probe tip 34, at its distal surface, includes one or more irrigation ports 18. The irrigation ports 18 are all connected to an internal irrigation passage, preferably centrally located in the probe tip 34 and the probe 20. In addition to the configuration shown in FIG. 4, the probe 20 can have, extending along its length, one or more grooves or channels for aspiration, as discussed in more detail below.

FIGS. 5 and 8 show features of the ultrasonic medical device of another embodiment of the present invention. As shown in FIG. 5, the ultrasonic treatment apparatus has an ultrasonic probe 20 with the ultrasonic tip 34. The ultrasonic probe 20 is housed in, for slidable movement within, a flexible articulation sheath 70. The flexible articulation sheath 70 is, in turn, housed in, for slidable movement within, a rigid sheath 80. The rigid sheath 80 is connected to, for movement with, a retracting housing 90. The retracting housing 90 is connected to a retracting trigger 94, which is pivoted on a handle 5. The retracting housing 90 may include an aspiration fitting or luer 13, which is configured for connection with a flexible tube which is in turn connected to a source of reduced pressure. The aspiration fitting or luer 13 is connected to the interior of the flexible articulation sheath 70.

An articulation trigger 91 may be housed on the retracting housing 90. Articulation trigger 91 is connected to an articulation wire 71. A trigger 92 may also be housed on the retracting housing 90. A cover 93 may cover components between the retracting housing 90 and the handle 5. FIG. 8 shows the details of the distal end 24 of the transverse mode probe 20 of FIG. 5. The ultrasonic probe 20 may include one or more grooves or channels 60 which are used to provide aspiration to the area around the probe tip 34. One or more irrigation lumens 61 may provide irrigating fluid to the area around the probe tip 34. The ultrasonic probe 20, which, because of its small cross-sectional profile and the material of which it is constructed, is somewhat flexible so that it may be bent or articulated. The ultrasonic probe 20 fits within, for axial movement, the articulation sheath 70, which is made of a relatively flexible and resilient material. The space 72 between the ultrasonic probe 20 and the articulation sheath 70, together with the grooves or channels 60, form aspiration passages. The articulation sheath 70 may include, at one or more locations around the circumference of the articulation sheath 70, one or more embedded articulation wires 71, with a distal end affixed to the articulation sheath 70. The proximal end of the articulation wire 71 is affixed to the articulation trigger 91. The articulation sheath 70 is housed within, for axial movement, the rigid sheath 80. The rigid sheath 80 is made of a relatively rigid material.

When the rigid sheath 80 is slid back away from the distal end of the articulation sheath 70, and the articulation wire 71 is pulled axially inwardly by the articulation trigger 91, the articulation sheath will bend or articulate in a bending or articulation direction A. As a result, the ultrasonic probe 20 and probe tip 34 will bend or articulate in articulation direction A. In this way, the ultrasonic probe 20 and probe tip 34 can be used to reach locations which are not axially aligned with the lumen or vessel through which the ultrasonic probe 20 is inserted.

FIG. 6 shows a longitudinal cross-section of a portion of the ultrasonic probe 20 and the probe tip 34 according to one embodiment of the present invention. The probe 20 includes a central passage 62, a plurality of lateral lumens 61, and a plurality of external aspiration grooves or channels 60. The central passage 62 of the probe 20 is connected to an irrigation fitting or luer 2. In a preferred embodiment, the central passage 62 terminates in two lateral lumens 61, located on the sides of the probe 20. The central passage 62 is used to transmit an irrigating fluid to the area around the probe tip 34, to thereby regulate the temperature of the treatment site. The irrigation fluid, together with the cavitational action of the probe tip 34, allows the treatment site to be regulated to a temperature of ±7° of normal body temperature. Furthermore, because the lumens 61 do not pass through the probe tip 34, the effective area of treatment of the probe tip 34 is increased.

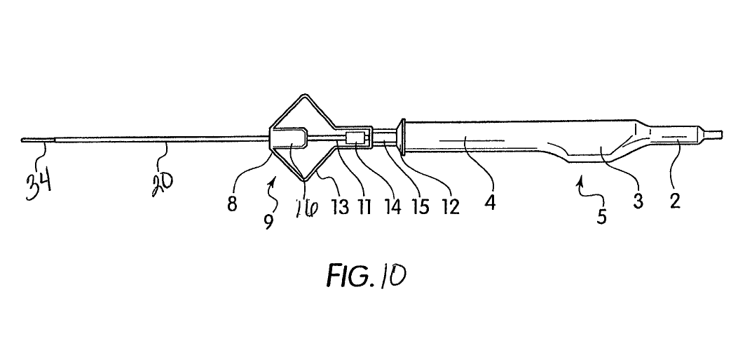

As shown in FIGS. 6 and 7, the outer surface of the ultrasonic probe 20 includes one or more grooves or channels 60. The grooves or channels 60, although straight in FIG. 6, could spiral along the length of the ultrasonic probe 20. The grooves or channels 60 are used to aspirate fluid and tissue fragments from the treatment site, as the result of negative pressure or suction applied at the proximal ends of the grooves or channels 60. As a result, fluid and tissue fragments travel down the grooves or channels 60 and away from the treatment site, thereby preventing fluid and fragments from interfering with the ultrasonic processing and cavitation of additional tissue.

FIG. 7 shows a transverse cross-section of a portion of the ultrasonic probe shown in to FIG. 6. In this embodiment of the present invention, the probe 20 includes a plurality of arcuate channels 60 that extend over the longitudinal length of the probe, providing a conduit for irrigation and or aspiration of tissue debris and fluid.

In another embodiment of the present invention, the probe comprises at least one aspiration channel, and aspiration of tissue debris is effectuated along the probe length between the interior surface of the sheath and the exterior surface of the probe, as directed by the aspiration channels.

In another embodiment, the device of the present invention comprises an irrigation channel that allows passage of fluids to the surgical site and facilitates removal of debris generated by action of the probe. The sheath is adapted to an irrigation means, and the sheath directs fluid to the location of the probe 20. The irrigation channel can be manufactured out of the same material as the sheath provided it is of a sufficient rigidity to maintain its structural integrity under the positive pressure produced by the flow of fluid produced by the irrigation means. Such an irrigation channel can be provided inside the lumen of the sheath, or along the exterior surface of the sheath, or the sheath itself may provide the aspiration channel. When the sheath itself is used to provide the irrigation, there is an added benefit that the probe is cooled by the irrigating fluid.

In another embodiment of the present invention, the sheath further comprises both an irrigation channel and an aspiration channel. As in the embodiments discussed above, the irrigation channel and the aspiration channel may be located within the sheath lumen, or exterior to the sheath, or a combination of the two. The sheath lumen itself may provide either the irrigation channel or the aspiration channel, with the corresponding irrigation channel or aspiration channel either contained within or external to the sheath.

In another embodiment of the present invention, the sheath comprises a means for directing, controlling, regulating, and focussing the cavitation energy emitted by the probe, an aspiration means, an irrigation means, or any combination of the above.

FIG. 9 shows another embodiment of the ultrasonic probe aspiration sheath or catheter of the present invention. In the embodiment of FIG. 9, a tip 75 of the aspiration sheath or catheter 100 is a rounded end. The aspiration sheath or catheter I 00 includes a lateral slot or opening 19 on one side. The ultrasonic probe 20, with the probe tip 34 which may optionally include a bevel 21, is mounted for axial sliding movement within the aspiration sheath or catheter 100. At least one aspiration passage 23 is created in the space between the ultrasonic probe 20 and the interior wall of the aspiration sheath or catheter 100. Accordingly, as suction is applied to the aspiration fitting or luer 13, a negative pressure or suction is formed at the aspiration passage 23, to draw away any destroyed or cavitated tissue and any residual or irrigation fluid.

At the proximal end of the tip 75 of the sheath 100 is a grasping surface or backstop 76. This grasping surface or backstop 76 serves as an opposed surface to the probe tip 34, thereby allowing dangling or loose treatment areas to be grasped during treatment. In operation, the aspiration sheath or catheter 100 is directed to a treatment area, until the dangling or loose treatment area falls into the lateral slot or opening 19. During this step, the ultrasonic probe 20 is in a retracted position, as shown in FIG. 9. Thereafter, the ultrasonic probe 20 is advanced axially outward, until the dangling or loose treatment area is clamped between the probe tip 34 and the grasping surface or backstop 76. Thereafter, the ultrasonic vibration generator is activated, such that ultrasonic energy is transmitted to the probe tip 34. As a result, the grasped treatment area is treated using ultrasonic energy and the resulting cavitation.

In another embodiment of the present invention, the sheath comprises a surface that is capable of manipulating the tissues near the site of the probe. The terminus of the sheath may be closed, such that the sheath insulates tissues from the energy emitted by the probe and can be used to push tissues away from the aperture, thereby allowing proximal tissues to be exposed to the probe and alternatively, the sheath comprises a beveled or arcuate surface at the sheath terminus capable of providing a means for hooking, grasping, or otherwise holding a tissue in proximity to the probe. In another embodiment of the present invention, the sheath provides a means for introducing a surgical device, for example, flexible biopsy forceps, capable of manipulating tissues into a tissue space, such that the surgical device can hold the tissue in proximity with the probe.

FIG. 10 shows an embodiment of the ultrasonic medical device 10 of the present invention, which includes the handle 5 and an aspiration shroud 9 for connecting the transverse mode probe to a source of negative pressure. The ultrasonic medical device 10 includes the ultrasonic probe 20 with the probe tip 34. The ultrasonic probe 20 is axially movably mounted within an aspiration sheath or catheter 100, so that the probe tip 34 may move axially inwardly and outwardly relative to the distal end of the aspiration sheath or catheter 100. The ultrasonic probe 20 and the aspiration sheath or catheter 100 are both mounted in an aspiration shroud 9, which includes an aspiration shroud housing 8. Within the aspiration shroud housing 8 is an aspiration end 16 of the aspiration sheath or catheter 100, which transmits suction or negative pressure to the interior of the aspiration sheath or catheter 100. The aspiration end 16 surrounds, and is sealed against, an ultrasonic transmission element 11 which extends to, and forms a proximal portion of, the ultrasonic probe 20. The aspiration end 16 is connected to an aspiration fitting or luer 13. The aspiration fitting or luer 13 is configured for connection with a flexible tube which, in turn, is connected to a source of reduced pressure. The aspiration sheath is slidable relative to the handle 5 and probe 20, thereby allowing the distance between the probe tip 34 and the distal end of the aspiration sheath or catheter 100 to be varied. The handle 5 is composed of an irrigation fitting or luer 2, a grasping area 3, and a probe fitting 4. The irrigation fitting or luer 2 is configured for connection with a flexible tube which is in turn connected to a source of pressurized irrigating fluid, such as water. The grasping area 3 is shaped for grasping by the hand of the apparatus operator, such as a surgeon, and may include one or more trigger or button mechanisms for activating and deactivating various features of the apparatus, such as suction, irrigation, power, etc. An actuation mechanism 12 may extend from the aspiration shroud 9 to the handle 5, and is surrounded by suitable covers 14, 15.

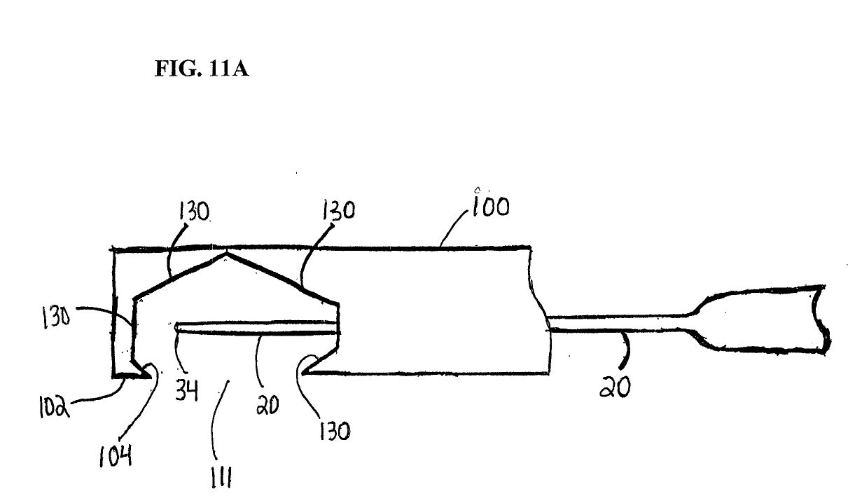

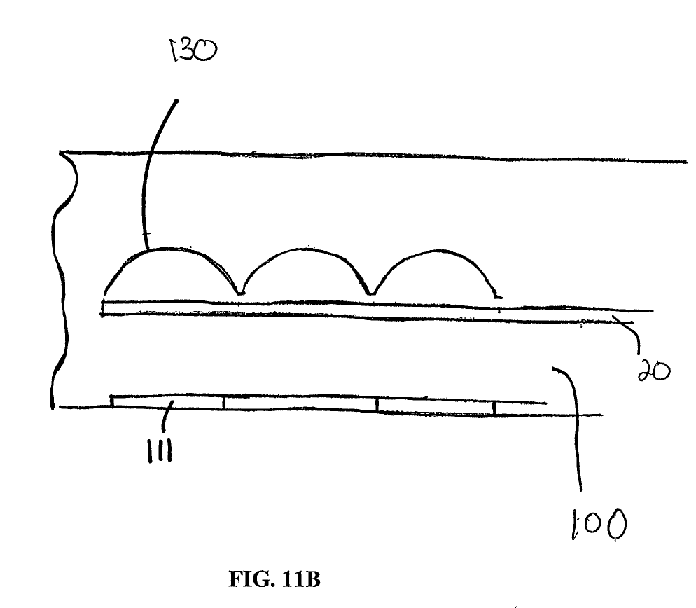

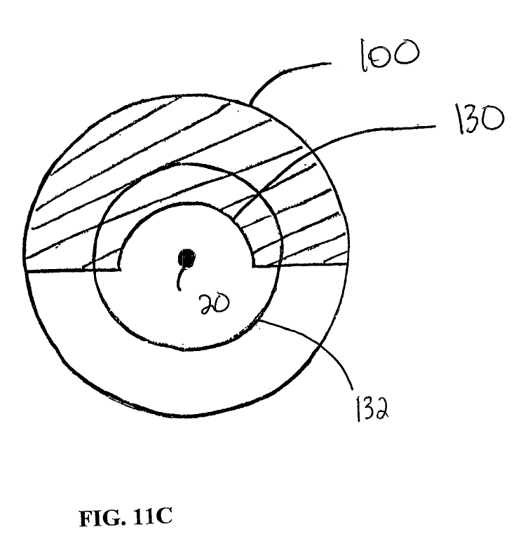

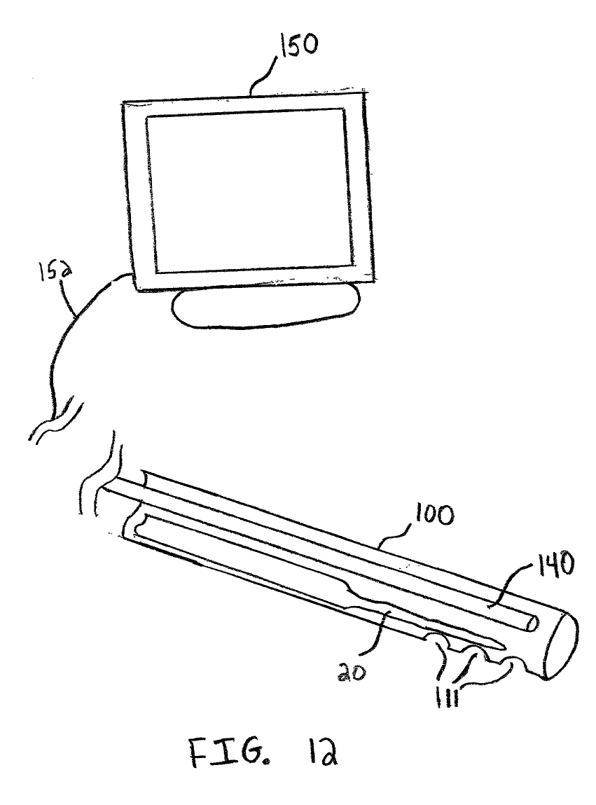

FIG. 11A shows an embodiment of the present invention wherein the probe 20 and the probe tip 34 are substantially contained within the sheath 100. The sheath 100 includes a plurality of fenestrations 111 allowing communication of the cavitation energy emitted by the probe 20 to areas outside of the sheath 100. The interior surface 106 of the sheath 100 further comprises a plurality of reflective elements 130, shown as a plurality of planar surfaces that extend from the interior surface 106 of the sheath 100 into the lumen, thereby providing a means for focusing and redirecting cavitation energy emitted by the probe 20. The reflective elements 130 shape can be any shape that reflects acoustic waves including, but not limited to, planar, triangular, or arcuate. In this embodiment of the present invention, the terminal end 102 of the sheath 100 is shaped to provide a tissue manipulation means and includes a bevel In. FIG. 11B shows another embodiment, wherein the reflective elements 130 are arcuate, and the sheath 100 further comprises a plurality of fenestrations 111. The arcuate shape of the reflective elements 130 corresponds to the shape and multiple locations of nodes 40 and anti-nodes along the probe 20. FIG. 11C is a sectional view of a transverse mode probe 20 of the present invention at least partially covered by an acoustic sheath 100 having at least one reflective element 130. As shown in FIG. 11C, a cavitation wave 132 expands from the probe 20. The area of the cavitation wave 132 shown in FIG. 11C is approximate and the cavitation wave 132 may expand to a larger or a smaller area than shown. The cavitation wave expands from the probe 20 until reaching the reflective elements 130 attached to the interior surface 106 of sheath 100. The reflective elements 130 then reflect the cavitation wave focussing and redirecting cavitation energy emitted from the probe 20.

FIG. 12 shows another embodiment of the present invention including a means of viewing the site of probe action which may include an illumination means and a viewing means. In an embodiment of the present invention, the sheath 100 includes a means for containing or introducing (if external to the sheath) an optical imaging element 140. The optical imaging element 140 may be any optical imaging element known to those skilled in the art, including, but not limited to, an endoscope or a similar viewing instrument with capabilities of diagnostic or therapeutic function. The viewing component of an endoscope is made up of numerous mini-light transmitting glass fibers bundled tightly together and has special channels. In an embodiment of the present invention, the ultrasonic medical device is used in conjunction with an imaging system, for example, using non-ferrous probes that are compatible with MRI, or ultrasound imaging, in particular, color ultrasound. In an embodiment of the present invention, the action of the probe 20 echogenically produces a pronounced and bright image on a display 150. The display 150 may be any display known to those skilled in the art, including, but not limited to, a CRT monitor, a LCD monitor or similar display. The optical imaging element 140 transmits the image of the probe 20 to the display 150 by an image data transmitter 152. The image data transmitter 152 may be any data transmitter known to those skilled in the art, including, but not limited to, a cord or similar data transmitter device. In this embodiment of the present invention, the sheath 100 shields the probe 20, thereby reducing the intensity of the probe image and enhancing the resolution of the surrounding tissues on the display 150.

In another embodiment of the present invention (not shown), the probe is used with an optical system. In one embodiment of the present invention, the probe is inserted into a body cavity or lumen along with a light-transmitting element for transmitting and receiving light from a light source, and transmitting the received light to a detector. The light from the light source (e.g., a laser) is transmitted through the light transmitting element, illuminating the area surrounding the probe 20, and the light transmitted back through the light transmitting element (e.g., from tissue in the vicinity of the probe) is detected by the detector. In one embodiment of the present invention, the light transmitting element is an optic fiber. In another embodiment of the present invention, the light transmitting element is a plurality or “bundle” of optic fibers. The light transmitting element can be a part of the probe or can be inserted into a body cavity independently of the probe. In one embodiment of the present invention, a sleeve is attached to the probe and the light transmitting element is held within the sleeve. In one embodiment of the present invention, a user serves as the detector (e.g., a physician or a medical technician). Light is monitored using a viewing element, such as an eyepiece (e.g., as in a microscope coupled to the light-transmitting element). It is preferred that the viewing element is not connected to a part of the ultrasonic medical device which is subject to vibration, to reduce manipulation of the viewing apparatus to a minimum. In another embodiment of the present invention, the detector is in communication with a processor, and converts optical signals from the light-transmitting element to data relating to the tissue in the vicinity of the probe.

In one embodiment of the present invention, the sheath assembly comprises an inner sheath and an outer sheath. The outer sheath may be connected to a retraction trigger, by one or more articulation means, such as wires, which is capable of moving the outer sheath with respect to the inner sheath. Each wire comprises a first end and a second end. The first end is affixed to the outer sheath, while the second end is affixed to a retraction trigger. When the outer sheath is slid back away from the terminus of the inner sheath the tissues are exposed to cavitation energy emitted by the probe. In another embodiment of the present invention, the first sheath is adapted to articulation wires. In this embodiment of the present invention, moving the sheath exposes the probe to a lumen of a second sheath, comprising a plurality of fenestrations which allow communication of the energy emitted from the probe to the lumen of a balloon catheter. Thus, a probe can be operational without inflating the balloon catheter until movement of the first sheath exposes the probe, thereby allowing the probe to penetrate occlusions that would otherwise prevent placement of the balloon catheter without first clearing a site for placement within the occlusion, and thereby reducing the number of steps in a surgical procedure.

In another embodiment of the present invention, the probe and sheath are flexible. Articulation wires comprising two ends are connected to the sheath and to an articulation handle respectively. When the articulation handle is manipulated, for example, pulled axially inward, it causes the flexible sheath to bend or articulate in a bending motion in the articulation direction. This way, the ultrasonic probe can be used to reach locations that are not axially aligned with the lumen or vessel through which the sheath and probe are inserted. One embodiment of the present invention uses such an articulable sheath to direct placement of a probe and a balloon catheter to a surgical site. This embodiment of the present invention is useful, for example, in clearing a blockage from the fallopian tubes.

In another embodiment of the present invention, the sheaths may be provided along with an ultrasonic probe in the form of a kit. The probe for a particular surgical procedure is provided along with the correct sheath, as well as instructions for assembling and tuning the probe, and the appropriate frequency range for the procedure. The probe and sheath may be packaged preassembled, such that the probe is already contained within the sheath and the respective position of the probe within the sheath is optimized such that any reflective elements in the sheath would be correctly aligned with the prospective position of the plurality of nodes 40 and anti-nodes 42 for a given frequency, the kit further comprising instructions for the appropriate frequency. The kit may further comprise packaging whereby the probe and sheath are presterilized, and sealed against contaminants. In another embodiment of the present invention, the probe and sheath is provided in a container that complies with regulations governing the storage, handling, and disposal of sharp medical devices. Such a container is capable of storing and dispensing the probe and additional adaptations such as sheath assemblies and balloon catheters prior to its use, and receiving and securing the probe and additional adaptations after use. A probe dispensation and disposal container of the present invention is the subject of the Assignee's co-pending patent application U.S. Ser. No. 09/775,908 entitled “Dispensation And Disposal Container For Medical Devices,” the entirety of which is hereby incorporated by reference. The dispensation and disposal container provides a means of affixing the probe and additional adaptations such as a sheath assembly to an ultrasonic medical device without direct manipulation of the probe and sheath assembly, and a means for detaching the probe and assembly from the ultrasonic medical device after use. In one embodiment of the present invention, the kit comprises a probe and sheath assembly contained within a sterile probe container that further comprises a single use locking means, whereby the probe and sheath assembly is affixed to the ultrasonic medical device solely through the container, and removed from the device after use solely through the container. The probe container also functions as a disposal container for the probe. Once the probe is removed from the container, the container design only allows for reinsertion of the extracted probe for disposal, and precludes re-extraction of the disposed probe, thereby discouraging multiple use of the probe.

The ultrasonic medical device 10 of the present invention can be used to treat a wide variety of gynecological diseases. A significant advantage of the present invention over the prior art is the ultrasonic medical device 10 operates in a transverse mode to cause fragmentation of diseased tissue without the excision of adjacent non-diseased tissue. Thus, the apparatus and method of the present invention is for treatment of gynecological diseases including, but not limited to: endometriosis, cysts, polyps, tumors, warty growths (i.e., condyloma acuminatum), or abnormal cellular growths or lesions of tissues of the female reproductive tract and surrounding areas. The present invention has particular application in removal of abnormal cell growths on the surface of the tissues of the female reproductive system, or organs such as the uterus or ovaries, including, but not limited to, leiomyoma (fibroids) such as intramural, subserous, and submucous leiomyoma, invasive epidermoid carcinoma, cervical intraepithelial neoplasia (CIN) grades 1, 2, and 3, adnexal mass of the fallopian tubes, ovarian tumors such as epithelial tumors, gonadal stromal tumors, germ cell tumors, endometrial polyps, endometrial hyperplasia, adenomateous hyperplasia of the endometrium, and carcinoma in situ of the endometrium. The present invention also has particular application in treating cervical cancer and related diseases that primarily affect tissues located on the surface of the uterus or cervix.

A method for treating gynecological diseases by destroying targeted cells on a surface of a body cavity includes inserting a member having a longitudinal axis into the body cavity, providing ultrasonic energy to the member and generating an area of cavitation along the longitudinal axis of the member, and sweeping the member over the surface of the body cavity to destroy the targeted cells. The sweeping the probe 20 of the ultrasonic medical device 10 over a target tissue creates a tissue-destructive effect within the vicinity of the probe 20. The method of the present invention can also include aspiration and irrigation of the treated tissue as discussed above. The sweeping of the probe 20 of the ultrasonic medical device 10 over a target tissue is preferably in a windshield-wiper fashion with the tissue removed in all areas adjacent to the plurality of anti-nodes 42 being produced along the entire length of the probe 20. Alternatively, the sweeping of the probe 20 may be in a longitudinal, spiral, or any other fashion necessary to destroy the target tissue. Once a lesion or growth has been ablated, the user of the probe (e.g., a physician or a medical technician) moves the ultrasonic probe to the next site having abnormal cells and repeats the process. It is anticipated that in a preferred method of treatment of the present invention, two or more cavitational treatments may be required.

By using the ultrasonic probe in conjunction with an imaging system, the user of the probe (e.g., a physician or a medical technician) uses information received from the imaging system to guide the movement of the probe. In this way, damage to tissues which do not comprise areas of abnormal growth can be minimized. Because the effects of cavitation on tissue are immediate, the user can discern the point of the procedure (e.g., the depth of tissue destruction and the effect that the procedure has had). In contrast, prior art methods such as cryosurgery and electrocautery produce effects several weeks after treatment (e.g., tissue sloughing) and thus the actual effect of the procedure can not be monitored at the time of treatment.

Variations, modifications, and other implementations of what is described herein will occur to those of ordinary skill in the art without departing from the spirit and scope of the present invention as claimed. Accordingly, the present invention is to be defined not by the preceding illustrative description but instead by the spirit and scope of the following claims.

What is claimed is:

1. A device for treating a tissue adjacent a body lumen comprising:
an elongated probe having a proximal end, a distal end, and a longitudinal axis wherein the elongated probe can be inserted into the body lumen; and
an ultrasonic generator for providing ultrasonic energy to the elongated probe for emission along the longitudinal axis of the elongated probe to the tissue adjacent the body lumen, wherein the ultrasonic energy creates a standing transverse wave in the elongated probe such that a plurality of nodes and a plurality of anti-nodes are formed along the longitudinal axis of the elongated probe to treat the tissue adjacent the body lumen.

2. The device of claim 1, wherein the tissue adjacent the body lumen includes abnormally growing cells.

3. The device of claim 1, wherein the body lumen is a uterus.

4. The device of claim 1, wherein the tissue includes cancer cells.

5. The device of claim 1, wherein the elongated probe is at least partially covered with a sheath.

6. The device of claim 5, wherein the sheath creates an aspiration passage between the elongated probe and the sheath.

7. The device of claim 5, wherein the sheath is moveable relative to the elongated probe.

8. The device of claim 5, wherein the sheath is flexible.

9. The device of claim 5, wherein the sheath is at least partially covered by an outer sheath.

10. The device of claim 5, wherein the elongated probe is coupled to a handle that controls the movement of the sheath.

11. The device of claim 1, further comprising a light transmitting element that can be inserted into the body lumen for obtaining optical data from the tissue.

12. The device of claim 11, wherein the optical data is an image of the tissue.

13. The device of claim 11, wherein the optical data is used to guide movement of the elongated probe.

14. The device of claim 11, wherein the optical data is a magnetic resonance image.

15. The device of claim 1, wherein an amount of ultrasonic energy to be applied to the tissue adjacent the body lumen is a function of an amplitude and a frequency of vibration of the elongated probe.

16. The device of claim 1, wherein the ultrasonic energy has a frequency that destroys water-laden tissues while not harming high-collagen connective tissue or other fibrous tissues.

17. The device of claim 1, wherein the ultrasonic energy has a frequency in the range of about 20,000 Hertz to about 80,000 Hertz.

18. The device of claim 1, wherein the ultrasonic energy has a frequency in the range of about 20,000 Hertz to about 35,000 Hertz.

19. A device for treating gynecological diseases by removing targeted cells on a surface of a body cavity comprising:
a member having a proximal end, a distal end, and a longitudinal axis; and
an ultrasonic generator for providing ultrasonic energy to the member and generating an area of cavitation along the longitudinal axis of the member to remove targeted cells on the surface of a body cavity.

20. The device of claim 19, wherein the targeted cells include abnormally growing cells.

21. The device of claim 19, wherein the body cavity is a uterus.

22. The device of claim 19, wherein the targeted cells include cancer cells.

23. The device of claim 19, wherein the ultrasonic energy creates a standing transverse wave in the member such that a plurality of nodes and anti-nodes are formed along the longitudinal axis of the member.

24. The device of claim 19, wherein the member is at least partially covered with a sheath.

25. The device of claim 24, wherein the sheath creates an aspiration passage between the member and the sheath.

26. The device of claim 24, wherein the sheath is moveable relative to the member.

27. The device of claim 24, wherein the sheath is flexible.

28. The device of claim 24, wherein the sheath is at least partially covered by an outer sheath.

29. The device of claim 24, wherein the member is coupled to a handle that controls the movement of the sheath.

30. The device of claim 19, further comprising a light transmitting element that can be inserted into the body cavity for obtaining optical data from the targeted cells.

31. The device of claim 30, wherein the optical data is an image of the cells.

32. The device of claim 30, wherein the optical data is used to guide movement of the member.

33. The device of claim 30, wherein the optical data is a magnetic resonance image.

34. The device of claim 19, wherein an amount of ultrasonic energy to be applied to the targeted cells on the surface of a body cavity is a function of an amplitude and a frequency of vibration of the member.

35. The device of claim 19, wherein the ultrasonic energy has a frequency that destroys water-laden targeted cells while not harming high-collagen connective tissue or other fibrous tissues.

36. The device of claim 19, wherein the ultrasonic energy has a frequency in the range of about 20,000 Hertz to about 80,000 Hertz.

37. The device of claim 19, wherein the ultrasonic energy has a frequency in the range of about 20,000 Hertz to about 35,000 Hertz.

38. A method of treating a tissue adjacent a body lumen, the method comprising:
(a) inserting a flexible member comprising a longitudinal axis into the body lumen; and
(b) providing ultrasonic energy from an ultrasonic generator to the flexible member for emission along the longitudinal axis of the flexible member to the tissue adjacent the body lumen.

39. The method of claim 38, wherein the tissue adjacent the body lumen includes abnormally growing cells.

40. The method of claim 38, wherein the body lumen is a uterus.

41. The method of claim 38, wherein the tissue includes cancer cells.

42. The method of claim 38, wherein the ultrasonic energy creates a standing transverse wave in the member such that a plurality of nodes and anti-nodes are formed along the longitudinal axis of the member.

43. The method of claim 38, wherein the member is at least partially covered with a sheath.

44. The method of claim 43, wherein the sheath creates an aspiration passage between the member and the sheath.

45. The method of claim 43, wherein the sheath is moveable relative to the member.

46. The method of claim 43, wherein the sheath is flexible.

47. The method of claim 43, wherein the sheath is at least partially covered by an outer sheath.

48. The method of claim 43, wherein the member is coupled to a handle that controls the movement of the sheath.

49. The method of claim 38, further comprising inserting a light transmitting element into the body lumen to obtain optical data from the tissue.

50. The method of claim 49, wherein the optical data is an image of the tissue.

51. The method of claim 49, wherein the optical data is used to guide movement of the member.

52. The method of claim 49, wherein the optical data is a magnetic resonance image.

53. The method of claim 38, wherein an amount of ultrasonic energy to be applied to the tissue adjacent the body lumen is a function of an amplitude and a frequency of vibration of the member.

54. The method of claim 38, wherein the ultrasonic energy has a frequency that destroys water-laden tissues while not harming high-collagen connective tissue or other fibrous tissues.

55. The method of claim 38, wherein the ultrasonic energy has a frequency in the range of about 20,000 Hertz to about 80,000 Hertz.

56. The method of claim 38, wherein the ultrasonic energy has a frequency in the range of about 20,000 Hertz to about 35,000 Hertz.

57. A method for treating gynecological diseases by destroying targeted cells on a surface of a body cavity, comprising the steps of:
(a) inserting a member having a longitudinal axis into the body cavity;
(b) providing ultrasonic energy to the member and generating an area of cavitation along the longitudinal axis of the member; and
(c) sweeping the member over the surface of the body cavity to destroy the targeted cells.

58. The method of claim 57, wherein the targeted cells include abnormally growing cells.

59. The method of claim 57, wherein the body cavity is a uterus.

60. The method of claim 57, wherein the targeted cells include cancer cells.

61. The method of claim 57, wherein the ultrasonic energy creates a standing transverse wave in the member such that a plurality of nodes and anti-nodes are formed along the longitudinal axis of the member.

62. The method of claim 57, wherein the member is at least partially covered with a sheath.

63. The method of claim 62, wherein the sheath creates an aspiration passage between the member and the sheath.

64. The method of claim 62, wherein the sheath is moveable relative to the member.

65. The method of claim 62, wherein the sheath is flexible.

66. The method of claim 62, wherein the sheath is at least partially covered by an outer sheath.

67. The method of claim 62, wherein the member is coupled to a handle that controls the movement of the sheath.

68. The method of claim 57, further comprising the step of inserting a light transmitting element into the body cavity for obtaining optical data from the targeted cells.

69. The method of claim 68, wherein the optical data is an image of the cells.

70. The method of claim 68, wherein the optical data is used to guide movement of the member.

71. The method of claim 68, wherein the optical data is a magnetic resonance image.

72. The method of claim 57, wherein an amount of ultrasonic energy to be applied to the targeted cells on the surface of the body cavity is a function of an amplitude and a frequency of vibration of the member.

73. The method of claim 57, wherein the ultrasonic energy has a frequency that destroys water-laden targeted cells while not harming high-collagen connective tissue or other fibrous tissues.

74. The method of claim 57, wherein the ultrasonic energy has a frequency in the range of about 20,000 Hertz to about 80,000 Hertz.

75. The method of claim 57, wherein the ultrasonic energy has a frequency in the range of about 20,000 Hertz to about 35,000 Hertz.

76. The method of claim 57, wherein the member is substantially flexible.

77. The method of claim 57, further comprising the step of aspirating fluids and debris from the targeted cells away from the area of cavitation.
